(19) United States
(12) Patent Application Publication
(10) Pub. No.: US 2024/0207298 A1
(43) Pub. Date: Jun. 27, 2024

(54) NUTRITIONAL SUPPLEMENTS FOR AMELIORATION OF RESPIRATORY TRACT INFECTIONS

(71) Applicant: Citrox Biosciences Limited, Kimbolton, Cambridgeshire (GB)

(72) Inventor: Richard Thomas, Kimbolton, Cambridgeshire (GB)

(73) Assignee: Citrox Biosciences Limited, Kimbolton, Cambridgeshire (GB)

(21) Appl. No.: 18/287,376

(22) PCT Filed: Apr. 19, 2022
(86) PCT No.: PCT/GB2022/050956

§ 371 Date: Oct. 18, 2023

(30) Foreign Application Priority Data
Apr. 19, 2021 (GB) 212105576.9

(51) Int. Cl. A61K 31/7048; A61K 9/00; A61K 9/20; A61K 31/593; A61K 31/724; A61K 31/728; A61K 36/752; A61P 31/14; A61P 37/06

(52) U.S. Cl. CPC A61K 31/7048; A61K 9/0043; A61K 9/0053; A61K 9/2009; A61K 9/2013; A61K 31/593; A61K 31/724; A61K 31/728; A61K 36/752; A61P 31/14; A61P 37/06

(57) ABSTRACT

The present invention relates to a nutritional supplement for use to ameliorate the effect of a pneumonia in a human, which supplement comprises a mixture of bioflavonoids which contains at least 45% wt/wt (of the total bioflavonoids) of naringin and at least 15% wt/wt of neohesperidin, wherein said supplement is in a form suitable for oral administration or administration to the lung by insufflation or inhalation. The present invention also provides a method of treatment of pneumonia using the aforementioned nutritional supplements.

### AREA OF THE INVENTION

The present invention relates to nutritional supplements containing a mixture of flavonoids suitable for use in the amelioration of respiratory tract infections such as pneumonia of bacterial or viral origin, such as that caused by SARS-COV-2 in humans. In particular, the present invention relates to the use of mixtures of flavonoids including naringin and neohesperidine, which may be administered to humans orally or to the lungs for the amelioration of pneumonia of bacterial or particularly viral origin, including the amelioration of pneumonia caused by SARS-COV-2. The nutritional supplement may also be employed to reduce the spread of SARS-COV-2. The present invention also provides a method of treatment of pneumonia using the aforementioned nutritional supplements.

### BACKGROUND TO THE INVENTION

Humans suffer from pneumonia of bacterial or viral origin. Thus, for example, humans are prone to pneumonias caused by influenza virus and coronavirus, of which SARS-COV-2 is of recent importance, having reached epidemic proportions.

When the pneumonia is of viral origin, the use of antiviral agents is only partially successful in humans, and benefits can be primarily a reduction in the term of hospitalization. Although antivirals for the treatment of influenza exists, hundreds of thousands of sufferers die each year, partly because of cost of the antiviral agents limiting their availability, and partly because they are often administered too late in the infection. Similarly, with coronaviruses such as SARS-COV-2 (also referred to as COVID-19), some antivirals have been shown to be at least somewhat beneficial in hospital settings, but they tend to require administration intravenously, which limits ease of use and increases costs. They also appear ineffective in already hypoxic patients.

Certain viral pneumonias also give rise to hyperimmune conditions, in which the body's own immune system starts attacking lung tissue, which increases the severity of damage to the lungs. This is sometimes referred to as a cytokine storm. In the case of infections by SARS-COV-2, it is often this secondary mechanism which leads to the need for artificial respiration and even death. This condition can occur even after the primary viremia has been brought under control by the body's innate and/or adaptive immune systems, so that treatment with conventional antiviral agents tends to be ineffective.

Naturally occurring flavonoids are known to exhibit antibacterial and antiviral effects on surfaces, including exterior surfaces of the human body. This is disclosed, inter alia, as WO 2008/009956, WO 2008/09958, WO 2010/089600, WO 2012/017186 and WO 2014/030005. In particular, Citrox™ bioflavonoid-containing mouthwashes and toothpastes are commercially available for use in the buccal cavity. They are generally labelled “not for internal consumption”. Another product containing Citrox™ bioflavonoids is “Cold and Flu Guard”, which may be sprayed into the mouth or nose to provide an antivirally effective barrier to viral ingress into the body. Neither product has been published as being effective in respect of SARS-COV-2, and neither product has been suggested for use by administration for the systemic treatment of pneumonia of bacterial or viral origin.

The Western scientific community has not traditionally turned to the use of nutritional supplements containing mixtures of bioflavonoids for the amelioration of infections, owing in part to difficulty in certainty of predicable absorption. However, it has now been discovered that nutritional supplementation with Citrox™ bioflavonoids can ameliorate pneumonias of viral origin. Since these bioflavonoids are effectively non-toxic and readily administered, their use offers a significantly different approach to the treatment of pneumonias.

Zakaryan et al, Arch. Virol., 2539-2551 (2017) and Gorniak et al, Phytochem. Rev., 241-272 (2019) review antimicrobial flavonoids, but do not refer to the mixtures described herein, although they discuss many other flavonoids.

Where flavonoids have been contemplated, they are generally derivable from grapefruit, for example from the seeds of a grapefruit. However, these have not been employed by systemic administration in humans; for example, they are not used orally or by administration to the lung by insufflation or inhalation for the treatment of pneumonia.

### SUMMARY OF THE INVENTION

The present invention relates to nutritional supplements for use in amelioration of pneumonia, and methods of amelioration of pneumonia using such supplements. The nutritional supplements are suitable for use in the amelioration of pneumonia and other respiratory tract infections of bacterial or viral origin in humans, including those caused by coronavirus such as SARS-COV-2.

The present invention provides a nutritional supplement for use to ameliorate the effect of a pneumonia, which supplement comprises a mixture of bioflavonoids which contains at least 45% wt/wt (of the total bioflavonoids) of naringin and at least 15% wt/wt of neohesperidin.

The present invention also provides a nutritional supplement for use to ameliorate a hyperimmune condition, such as those associated with pneumonia caused by SARS-COV-2, which supplement comprises a mixture of bioflavonoids which contain at least 45% by weight (of the total bioflavonoids) of naringin and at least 15% wt/wt of neohesperidine.

The present invention also provides a nutritional supplement for use in reducing development of SARS-COV-2 infection in mildly affected humans.

The preceding nutritional supplement for use and the preceding method favourably are employed in respect of human viral pneumonia caused by SARS-COV-2.

The mixture of flavonoids present in the nutritional supplement will include naringin and neohesperidine. They make up at least 60% wt/wt of the total flavonoids present, and preferably at least 75% wt/wt of the flavonoids present. Naringin should be present in at least 40% wt/wt, and more aptly at least 50% wt/wt, of the flavonoids present. Neohesperidine should be present in at least 15% wt/wt, and more aptly at least 20% wt/wt, of the flavonoids present. Other flavonoids present are most suitably derivable from oranges, and in particular from the pith of bitter oranges, and preferably from the pith of the immature bitter orange.

It is very suitable that the nutritional composition also comprises residual biomass from the orange source. Such biomass can aid in solubilization of the flavonoids, which can be of assistance in formulation of the nutritional supplement. This is particularly apt when employing oral administration.

The flavonoids mixture may also contain other non-flavonoid compounds of the extract from an edible plant source, for example from a further citrus. Similarly, the nutritional supplement may use carbohydrates from the fruit providing the flavonoids. Thus, the nutritional supplement may also comprise other biomass from the pith of the bitter orange. This enables the avoidance of complex extraction processes, which would be required if individual pure flavanones needed to be prepared and then blended, and can lead to a more readily solubilized mixture of flavonoids.

If desired, the compositions for oral administration may also comprise naturally derivable acids such as citric acid, lactic acid, tartaric acid, malic acid, caprylic acid, ascorbic acid, acetic acid, and the like.

The nutritional supplement may also comprise a buffering agent, especially if provided in liquid form or in a dispersible or dissolvable solid form prior to ingestion, when a pH of 3-8, for example 4-7, is apt in the solution or dispersion which is then taken.

The nutritional supplement may also comprise agents to help solubilisation. Thus, for example, agents such as cyclodextrins (such as beta cyclodextrin or hydroxypropyl cyclodextrin) and/or glycerol and/or surfactant may be present.

Preferred mixtures of flavonoids and their optional biomass are described in WO 2008/009958, and are set forth for ease of reference in Reference Examples 1 and 2 herein. These mixtures have been commercially employed as Citrox™ mouthwashes and toothpastes, and are also described for use for sanitizing hand gels and even for sterilization of surfaces, including those in hospitals and ambulances by misting. None of these uses have recommended ingestion of Citrox™ products, unlike the present invention, which indicates their systemic use, for example by swallowing or by insufflation/inhalation to reach the lungs when used in infections such as pneumonia in humans.

Hence in one favoured aspect, the invention provides the compositions described herein for administration by swallowing (i.e. oral administration).

It has now been discovered that oral administration (and by extension insufflation /inhalation so as to reach the lungs) of the mixtures of flavonoids described herein is effective in ameliorating infective pneumonias, for example when added to the foodstuff or drinking water of chickens, employed as a model system of coronavirus infection. The mixtures of flavonoids described herein have also been found to be effective in cell lines to reduce SARS-COV-2 infectivity which demonstrates to the skilled person the suitability of their use in respect of infections by SARS-COV-2.

Similarly, the mixtures of flavonoids described have been found to reduce damaging cytokine release caused by coronavirus infection, thereby further confirming their suitability in treating infections with SARS-COV-2, since, as demonstrated herein, they have antiviral properties against that virus, as well as improving cytokine production.

### DETAILED DESCRIPTION OF THE INVENTION

The invention will be understood by the skilled person by reading to the claims hereinafter set forth and by reading the Reference Examples and Examples set forth hereinafter and the following description.

Bioflavonoid products are known to possess antibacterial and antiviral properties against a broad range of bacteria and viruses, although they have not before been demonstrated to be effective in ameliorating bacterial or viral infections of the lower respiratory tract such as lung infections, for example pneumonia. Nor have they been demonstrated to be effective in ameliorating lung infections caused by SARS-COV-2 in humans.

It has further been found that when tested in cell systems, the flavonoids described

herein in the form of those set out in Reference Example 3 reduce the viability of SARS-COV-2 and can kill bacteria associated with pneumonia in humans.

It will therefore be understood by the skilled worker that the invention herein provides a method of ameliorating a bacterial or particularly viral pneumonia in a human which comprises administration to the human in need thereof of an effective amount of a nutritional supplement comprising a mixture of bioflavonoids as described herein. The administration to a human may aptly be by insufflation/inhalation or, more favourably, orally.

The invention is particularly concerned with amelioration of SARS-COV-2 infection in humans, including amelioration of SARS-COV-2-induced pneumonia and hyperinflammatory conditions.

The amelioration may take the form of treatment of an existing respiratory tract disease or prophylaxis to reduce the likelihood of development of a respiratory tract disease caused by SARS-COV-2.

In the treatment of SARS-COV-2, it has become established to employ dexamethasone on hospitalised patients exhibiting symptoms of hypoxia, such as shortness of breath or measured oxygen saturation of less than 94%, for example less than 92%, 90% or 88%. It is believed that dexamethasone works by reduced pro-inflammatory cytotoxins released as a result of overstimulation of the immune system by the virus or remnants thereof, which may persist even after the primary virus is controlled or largely controlled by the immune system. Dexamethasone lacks significant antiviral activity, and is not suggested for use until hypoxia occurs (for example, when the patient requires supplementary oxygen), partly because it can also inhibit desirable inflammatory effects.

It has been discovered that the nutritional supplement for use for the purposes of amelioration of SARS-COV-2 pneumonia, as it not only possesses a direct antiviral effect against the virus, but also causes release of desirable antiviral cytokines while suppressing undesirable pro-inflammatory cytokines. Hence the nutritional supplements described herein are contemplated for use in reducing immune dysfunction induced by SARS-COV-2. Hence use of the nutritional supplement described herein (unlike dexamethasone) supplement described herein can produce an amelioration of SARS-COV-2-caused pneumonia at early and late stages of the disease. In particular, the present invention provides a nutritional supplement as described herein for the amelioration of pneumonia in infected persons who exhibit symptoms of hypoxia or who have exhibited symptoms of infection for at least 4, 5 or 6 days, including those who have exhibited symptoms of infection for at least 4, 5 or 6 days with subsequent diminution of infection. Treatment of this last class of patients is included because it is known that hyperimmune-driven pneumonia can occur subsequently to apparent improvement of symptoms; for example, after the initial viremia has abated as a result of antiviral action of the immune system.

With SARS-COV-2 infection, it is known that some patients require readmission to hospital even after discharge because of a later onset of a hypoimmune condition which leads to hypoxia and even death. This invention contemplates the oral administration of a nutritional supplement described herein to patients at and following discharge from hospital in order to ameliorate the occurrence of hypoimmune-caused pneumonia likely to lead to rehospitalization. This is particularly desirable, since up to about 20% of readmitted patients die.

Hence in one aspect the present invention provides the use of an effective amount of a nutritional supplement described herein for oral administration to a patient discharged from a hospital after having had SARS-COV-2 pneumonia in order to reduce the risk of readmission, said administration to continue for at least 4 days post-discharge. Aptly, administration will continue for at least 7, at least 10, at least 14 or at least 21 days post-discharge. Administration will generally not be required more than 28 days post-discharge, although in view of its non-toxic nature, continuation of administration may be continued for longer if desired as a precaution.

Agents such as dexamethasone are not yet normally given for this purpose, possibly because of concerns regarding reduction of desirable cytokine release, which can accompany the reduction in undesirable cytokine release. However, this is of reduced concern with the nutritional supplements described herein in view of their antiviral properties.

Numerous persons infected by SARS-COV-2, including those who have been hospitalized, continue to exhibit significant adverse effects for an extended period after the active infection is brought under control (sometimes referred to as “long COVID”).

Administration of the nutritional supplements described herein during the active phase and/or after the acute phase has been overcome by the body's immune system is contemplated as aiding the amelioration of such long-term effects and/or shorten the period during which such effects occur. Early use of the nutritional supplement described herein, for example commencing 1, 2, 3, or 4 days or longer after first symptoms appear, may reduce severity of the viremia, and so protect cells from damage leading to long-term effects of infection. Treatment may continue after acute symptoms abate, for example for at least 4, 7, 10, 14 or 21 days or more if desired.

The mixtures of flavonoids described herein may be considered a nutritional

supplement, as the flavonoids are present in foodstuffs. However, for most people, if not all, a normal diet does not provide sufficient flavonoids for the desirable effects set out herein to occur. Hence the compositions described herein may be used to supplement the usual diet. Because of their non-toxic nature, the nutritional supplement may be employed on a regular basis, somewhat analogously to how some people choose to take vitamin supplements.

Alternatively, the nutritional supplement may be taken when concern arises about the likelihood of SARS-COV-2 pneumonia or the like being contracted, or after the disease is diagnosed.

For human use, orally administered nutritional supplements may provide, for example, 0.25-7.5 g of the mixture of bioflavonoids per day, more usually 0.5-5 g per day, for example 1, 2, 3, 4 or 5 g per day. The nutritional supplement may typically be taken on, for example, up to 6 occasions a day, such as 1, 2, 3, 4, 5 or 6 times a day, and more aptly 2, 3 or 4 times a day.

If, in solid form, unit doses may be provided which contain, for example, 100 mg-2000 mg of the mixtures of flavonoids, such as about 250 mg, 300 mg, 400 mg, 500 mg, 600 mg, 700 mg, 750 mg, 800 mg, 900 mg or 1000 mg.

Apt solid unit dosage forms include capsules, tablets and sachets containing powder or granulates.

Unit dosage capsules are usually hard gelatine capsules containing an amount of a mixture of flavonoids, for example as set out in Reference Examples 1 or 2 herein. Said capsules generally contain 250-2000 mg, for example 500-1000 mg, of flavonoids, as larger capsules are less convenient to use.

If larger amounts are required, unit doses may be such that dissolution in water or another beverage may be employed; for example, the contents of a sachet may be dissolved in a convenient amount of water or beverage. Similarly, larger-content tablets will generally be readily splitable, or more aptly, will be readily dispersible or dissolvable in water or beverage. Effervescent tablets are particularly suitable, as they permit ready solution of the nutritional supplement in water or beverage for ease of use. Chewable tablets may also be employed.

Alternatively, the mixture of flavonoids may be provided already in solution form. This may be as sachets or the like containing the required unit dose. This itself can be further diluted with water or beverage if desired. It is also suitable that the mixture of flavonoids is present in a multidose container, such as a bottle, from which a measured dose can be withdrawn to provide the desired amount of flavonoids, which can then be further diluted with water or beverage if desired. Hence the concentration of flavonoids in the multidose solution may be such that a measuring spoon of set size, such as 5 ml or 10 ml, may be used to provide the required dose, or the bottle cap may be of an appropriate volume to be employed to provide the required unit dose.

Both readily dispersing and effervescent tablets may be made by employing conventional tabletting agents used in preparation of such tablets.

The nutritional supplement may also be delivered to the respiratory tract.

When administration is intended to ameliorate a hypoxic pneumonia, administration may be directed to the lungs. Thus, administration may occur by insufflation or inhalation. Generally, insufflation will be preferred, and administration will usually employ an insufflation device which delivers a mist of an aqueous solution of the mixture of flavonoids or a dry powder of the nutritional supplement described herein. The insufflation device may be conventional, and a number of devices are commercially available. It is apt that the device is one which senses the subject's breathing and administers the nutritional supplement to coincide with inward breaths, for example as used to deliver a medicament to asthmatics.

The nutritional supplements herein can be taken by humans on a regular basis to assist in the prevention of developing a pneumonia or hyperimmune condition. Orally administrable forms are particularly suitable for such uses.

Hence, particularly in the winter months, when lung infections are more common, the nutritional supplements may be taken to assist in the reduction of bacterial or particularly viral lung infections, including those due to influenza, parainfluenza, and coronaviruses (including SARS-COV-2 and the like).

For this purpose, the nutritional supplements comprising flavonoids may further include a vitamin such as Vitamin C, and particularly Vitamin D (especially D3 and 25-hydroxy D3), which may further assist subjects having otherwise lower than desired vitamin levels, and so further assist in reducing pneumonia.

In view of the experimental results set out herein, it is possible that one mechanism by which taking the nutritional supplements helps reduce hyperimmune conditions (including that which may occur following pneumonia, such as that caused by SARS-COV-2) is by causing a reduction in nuclear factor kappa B or MAPK pathway effectiveness, which leads to a reduction in the production of pro-inflammatory cytokines in a so-called cytokine storm in late serious disease.

Hence use of the nutritional supplements herein may aid in amelioration of serious viral pneumonias by two distinct mechanisms, namely an anti-viral effect plus a reduction in pro-inflammatory cytokines which could lead to hyperinflammatory conditions as a result of the immune system dysfunction.

When used for the amelioration of hyperimmune conditions, the compositions described herein may be employed even and particularly aptly after the initial viremia has been brought under control. Hence in the case of SARS-COV-2, administration may be continued or commenced after initial symptoms abate.

A composition which may be used to reduce transmission of SAR-COV-2 is available commercially as “Cold and Flu Guard” spray, which delivers flavonoids as in Reference Example 3 and hyaluronic acid to the nose and throat on spraying. This device containing such flavonoids has not hitherto been indicated as effective with respect to SARS-COV-2, but in view of the data in Reference Example 3, the skilled person will understand how it may be employed to reduce transmissions of SARS-COV-2. Such sprays may be employed as a ration, for example from 1-6 times a day, such as 2, 3 or 4 times a day. The spray may also be applied shortly before or shortly after an anticipated exposure to SARS-COV-2, for example 15 minutes before or after such a possible exposure.

The present invention also provides a nutritional supplement comprising bioflavonoids as hereinbefore described in a form suitable for intranasal administration to ameliorate infection by SARS-COV-2, or to treat nasopharyngeal infection by SARS-COV-2 and/or to reduce release of SARS-COV-2 particles on the breath from the nasopharyngeal areas. Thus, the compositions may be used to reduce transmission of SARS-COV-2 from an infected person and lower the incidence of infection in a community.

If desired, the nutritional supplement may be referred to as a “pharmaceutical composition”.

The nutritional supplement is aptly presented in a nasal applicator, from which a spray of droplets, for example as a fine mist, can be introduced into the nose. This may be accompanied by the subject inhaling to assist the droplets reaching the anterior and preferably also the posterior nasal chambers.

Many nasal applicators are commercially available, for example where squeezing the body of a plastic applicator releases a stream of droplets into the nose when the opening of the applicator is placed in or at the entrance to the nostril.

Generally, 50-150 μg may be administered twice to each nostril, for example 100 μg may be sprayed twice into each nostril. This may be performed 1, 2, 3, 4, 5 or 6 times a day, particularly 3 or 4 times a day, for example at breakfast, lunch and dinner times.

The nutritional supplement is aptly in the form of an aqueous solution comprising 0.25% wt/wt to 7.5% wt/wt, for example 0.5% wt/wt to 5% wt/wt, such as 1% wt/wt to 2.5% wt/wt of the bioflavonoids.

The solution may preferably further comprise one or more soluble polysaccharides which help immobilize and/or deactivate SARS-COV-2. Such polysaccharides may contain an ionizable moiety. Such moieties may be an acid or basic moiety such as a carboxylic acid or sulphonic acid, or an amino group, for example a dimethylamine alkyl group.

Suitable polysaccharides include glyosoaminoglycans such as heparin, heparin sulphate, hyaluronates such as hyaluronic acid, chitosan and the like.

Hyaluronic acid, for example as sodium salt, is particularly apt for use in the intranasal compositions.

Suitable polysaccharides also include sulphated polysaccharides such as carrageenans, for example iota-carrageenan and the above sulphated glycosaminoglycans. lota-carrageenan is a particularly apt sulphated polysaccharide for use in intranasal use of the nutritional supplements herein.

Such polysaccharides may be linear or branched chain.

Other suitable polysaccharides include cyclodextrins such as betacyclodextrin and hydroxypropyl cyclodextrin.

The polysaccharides may be employed in an amount so as to thicken the composition so that it resembles the constitution of normal nasal secretions when employed as part of the intranasally administrable composition. The molecular weights and concentrations of the polysaccharides may be chosen to this end.

The intranasal compositions will aptly employ a buffer to retain their pH in a range of 2.5-7.5, more aptly 3.5-6.5, and preferably 3.5-4. Any convenient intranasally available buffer may be employed that results in the preceding pH values. Thus, for example, mixtures of citric acid and/or tartaric acid and/or lactic acid and/or caprylic acid, together with their alkali metal salts, such as their potassium or sodium salts, may be employed, as may phosphate buffers employing alkali metal salts of phosphates. Once the skilled person is directed as herein to the appropriate pH, the buffer is readily provided in accordance with standard practice. Citrate buffers are believed to be particularly apt (i.e. mixtures of citric acid and a salt thereof, such as the sodium salt).

The intranasal compositions may also comprise an agent to assist in solubilization of the bioflavonoids. Suitable agents include polyhydroxy compounds such as glycerol, or other suitable agents such as surfactants. Suitable surfactants include non-toxic surfactants, such as those employing polyoxyethyleneglyol residues. Such surfactants include poloxamers, which are block copolymers of polyethyleneoxide (PEO) and polypropyleneoxide (PPO) arranged in a PEO-PPO-PEO form. Such poloxamers include Pluronics™. Other non-ionic surfactants include those with a hydrophilic head (such as in sugar residue) and a hydrophilic tail (such as a carboxyl acid residue). Such surfactants include those known as Brig™, Span™ or Tween™, and include sucrose esters such as laurate ester.

The intranasal composition may further comprise additional agents useful in deactivating SARS-COV-2. Such agents can preferably include xylitol and/or nitrite salts such as sodium nitrite or potassium nitrite.

The intranasal compositions may be administered to reduce spread of SARS-COV-2. Thus, for example, they may be administered to unvaccinated subjects, or to subjects who have received the first dose of a two-dose vaccine. The oral compositions described herein may similarly be employed to reduce the spread of SARS-COV-2, especially from or to such subjects. The intranasal composition may also be administered to subjects at risk of infection by SARS-COV-2 to reduce that risk. Such administration may be to subjects who have been informed of a risk (for example, exposure to an infected person) or to those who simply wish to reduce risk out of a care for personal and common good in reducing spread.

The invention also provides the use of an intranasal composition as described herein for use in reducing the time of shedding of SARS-COV-2 by a subject having a nasal SARS-COV-2 infection. This method of reducing shedding of SARS-COV-2 employs an effective amount of an intranasally administrable composition as described herein. Administration can be as hereinbefore described and continued for, for example, 1-7 days, more suitably 2-5 days, for example 3 or 4 days.

In another aspect, the invention provides a method for organ failure due to a cytokine storm (hyperinflammatory condition, for example of the liver, kidneys, and/or heart) which comprises administration to a person in need thereof an effective amount of a nutritional supplement as described herein. Such administration is preferably orally. Similarly, the present invention provides a nutritional supplement as hereinbefore defined for use in treatment of organ failure due to a cytokine storm (hyperinflammatory condition, for example of the liver, kidneys, and/or heart). The administration of the supplement may be orally. The cytokine storm may be caused by infection with SARS-COV-2 or otherwise.

When used to ameliorate pneumonia of bacterial origin, the nutritional supplement may be employed/administered as described hereinbefore with respect to viral pneumonia such as that caused by SARS COV-2.

The nutritional supplement used to ameliorate pneumonia due to SARS COV-2 as hereinbefore described may favourably be used to ameliorate infections caused by the omicron variant.

The use of a nutritional supplement as described hereinbefore to treat an infection by SARS COV-2 has the benefit of reducing the probability of the subject developing a pneumonia of bacterial origin as a subsequent infection.

A bioflavonoids comprising composition of Reference Example 3 was found to kill Streptococcus pneumoniae (an organism known to cause pneumonia in humans) when tested in vitro in two different conditions; solid growth media (blood agar plates) and liquid growth media. In both cases the composition was found to be very effective in both overnight solid culture conditions, and in short-term (5 h) and in long-term (24 h) liquid culture conditions, by killing bacteria even at the highest tested CFU/ml, ranging from 1×10² to 1×10⁸ CFU/ml (higher CFUs than what would normally cause infection in humans). The demonstration of such bactericidal effectiveness in both solid and liquid culture conditions across such a wide range of bacterial CFUs indicates that the bioflavonoids composition will be equally effective in vivo, e.g., when administered to humans either orally, or by insufflation or inhalation.

### Reference Example 1

A suitable supplement may contain bioflavonoids from bitter oranges, as follows:

| | % of Total Bioflavonoid |
|---|---|
| Bioflavonoid | Component |
| Isocriocirm | 2.4 |
| Isonaringin | 2.7 |
| Naringin | 52.0 |
| Hesperidine | 3.1 |
| Neohesperidine | 27.8 |
| Neodiomin | 3.1 |
| Naringenin | 3.4 |
| Poncirin | 4.4 |
| Rhiofolen | 1.1 |
| Total | 100% |

### Reference Example 2

A suitable supplement may contain bioflavonoide as set forth in Reference Example

1, together with other biomass derived from the pith of immature bitter oranges in the wt/wt ration of 5:1 to 1:5, such as 2:1 to 1:2, and most aptly 1.25:1 to 1:1.25, and preferably 45:55. A preferred description is:

| | % in total |
|---|---|
| Bioflavonoid | biomass |
| Isocriocirm | 1.1 |
| Isonaringin | 1.2 |
| Naringin | 23.4 |
| Hesperidine | 1.4 |
| Neohesperidine | 12.5 |
| Neodiomin | 1.4 |
| Naringenin | 1.5 |
| Poncirin | 2.0 |
| Rhiofolen | 2.8 |

### Reference Example 3

Liquid Formulation of Bioflavonoids

| | |
|---|---|
| Bioflavonoid of | 5% |
| Reference Example 2 | |
| Malic Acid | 15% |
| Citric Acid | 15% |
| Lactic Acid | 20% |
| Glycerine | 20% |
| Water | 25% |

### Reference Example 4

A suitable formulation for intranasal application is as follows:

| No. | NAME of RAW MATERIAL | % |
|---|---|---|
| 1 | WATER DISTILLED | 73.980 |
| 2 | ETHANOL 96% | 1.000 |
| 3 | GLYCERINE | 10.000 |
| 4 | COMPOSITION OF | 5.000 |
| | REFERENCE EXAMPLE 3 | |
| 5 | XYLITOL | 2.000 |
| 6 | STEVIA | 0.080 |
| 7 | DISODIUM PHOSPHATE | 1.800 |
| 8 | CETYLPYRIDINIUM CHLORIDE | 0.050 |
| 9 | POLYSORBATE 20 | 1.500 |
| 10 | MENTHOL crystals | 0.200 |
| 11 | WATER distilled | 1.000 |
| 12 | SODIUM HYALURONATE | 0.010 |
| 13 | SODIUM HYDROXIDE, 10% | 3.380 |
| | total: | 100.00 |

### Example 1

In this example, uninfected chicks are anticipated as reaching a weight of 1.5 kg at 30 days. The birds were infected with IBV at 14 days. Administration of the flavonoids commenced at day 22 (i.e. 8 days past infection). A relatively more concentrated solution of flavonoids was employed at a rate of 21 per 1001 of water. The solution was as set out in

Reference Example 3 herein, with the addition of 2 g/l of maltodextrin. This provided the chickens with 0.05 kg/day of a mixture of bioflavonoids.

### Illustration of Anti-Viral Effect

### Viruses, Media and Cells

Virus stocks were prepared prior to testing by growing hCoV-229E virus stock in Huh7 cells. Culture test media was MEM with 5% foetal bovine serum and 50 μg/mL gentamicin for HCoV viruses, PIV-3, and HRV viruses.

### Virucidal Assay

Reference Example 3 bioflavonoids concentrate solution was diluted in sterile water to test concentrations of 5%, 2% and 1%. Compounds were mixed directly with virus solution in three tubes at a volume ratio of 90% prepared compound and 10% virus solution. Test media only was added to one tube of each prepared concentration to serve as toxicity controls. Ethanol (70%) was tested in parallel as a positive control and water only as a virus control.

Solution and virus were incubated at room temperature for 1 minute. The solutions were then neutralized by a 1/10 dilution in test media.

Virucidal efficacy of Reference Example 3 bioflavonoids concentrate against hCoV-229E after a 1-minute contact time with virus at 22±2° C. was as follows:

| | | Test | | | | | |
|---|---|---|---|---|---|---|---|
| | Cell | Concen- | Cyto- | Neutralization | Virus | VC | |
| Virus | Type | tration | toxicity | Control | Titer | Titer | LRV |
| hCoV- | Huh7 | 5% | None | None | 2.3 | 3.0 | 0.7 |
| 229E | | | | | | | |
| hCoV- | Huh7 | 2% | None | None | 2.7 | 3.0 | 0.3 |
| 229E | | | | | | | |
| hCoV- | Huh7 | 1% | None | None | 3.0 | 3.0 | 0 |
| 229E | | | | | | | |

### Example 2

A test was performed as in Example 1 with a 1% solution of Reference Example 3 bioflavonoids concentrate with contact time of 5 minutes. Under these conditions, only 5% of virus remained infective. Such a 95% reduction in viral load would be considered very advantageous in vivo. Longer contact times were not employed, but it is anticipated that longer contact times will yet further reduce viable virus levels.

### Example 3

Tests analogous to those of Example 1 were performed with parainfluenza virus 3, influenza A (H1N1) and human respiratory virus 14, with 1% Reference Example 3 bioflavonoids concentrate. The results were as shown:

| | Cell | Virus | VC | |
|---|---|---|---|---|
| | Type | Titer | Titer | LRV |
| PIV-3 | mA-104 | <0.7 | 5.0 | 3.5 |
| Flu H1N1 | mDCK | <0.7 | 2.7 | 2.0 |
| HRV14 | HeLa | 1.5 | 5.0 | 3.5 |

Hence virucidal activity occurs against these potentially pneumonia-causing viruses also.

### Example 4

Treatment of Coronavirus Pneumonia Demonstrating, Inter Alia, Cytokine Modulation

A suitable formulation for intranasal application is as set forth in Reference Example Example 4.

### Example 5

In this example, uninfected chicks are anticipated as reaching a weight of 1.5 kg at 30 days. The birds were infected with IBV at 14 days. Administration of the flavonoids commenced at day 22 (i.e. 8 days post-infection). A relatively more concentrated solution of flavonoids was employed at a rate of 2 liters per 100 liters of water. The solution was as set out in Reference Example 3 herein, with the addition of 2 g/l of maltodextrin. This provided the chickens with 0.05 kg/day of a mixture of bioflavonoids.

Infectious bronchitis of chickens is a serious disease caused by coronavirus. The disease is characterized in the later stages by hyperinflammation of the lung due to the release of proinflammatory cytokines by the adaptive immune system. Vaccines are available against this coronavirus disease, but are not completely effective, with about 35% of chickens dying despite having been vaccinated when infected with the virus. It was observed that when chickens were challenged with the virus and a mixture of flavonoids as set out in Reference Example 3 added to their food for five days past challenge, the mortality rate decreased to 6%. Hence about 4 in every 5 birds were prevented from dying by use of a mixture of bioflavonoids as described herein. In addition, the general health of surviving birds was significantly better in the treated group as opposed to untreated group. Thus, for example, dyspnea was reduced from 17% to 7%, elevated pulse rate reduced from 28% to 4%, cough from 18% to 3%, sneezing reduced from 14% to 3%, ruffled feathers reduced from 28% to 4% and Tr rate reduced from 31% to 2%. Hence flavonoid-treated infected birds had a significantly reduced level of clinical symptoms compared to untreated infected birds. When viral load was determined in lung and trachea, viral DNA was reduced from about 7 (Log 10 viral RNA copies per gram of tissue) to about 1 in the lung and from about 8 to about 0.5 in the trachea.

The levels of post-inflammatory cytokines were very much reduced in the flavonoid-treated birds compared with the untreated birds. TNFα reduced from about 120 pg/ml to about 50 pg/ml (level in uninfected birds about 30 pg/ml) and TGF-β3 reduced from about 400 pg/ml to about 30 pg/ml (level in uninfected birds about 80 pg/ml). Similar reductions were noted in tracheal tissue. Interferon levels normalized in both tissues. In infected birds,

INFα reduced from about 500 pg/ml to about 50 pg/ml (compared to about 80 pg/ml in uninfected birds), INFβ reduced from about 1500 μg/ml to about 100 pg/ml (compared to about 200 pg/ml in uninfected birds) and INFγ reduced from about 100 pg/ml to about 20 pg/ml (compared to about 30 pg/ml in uninfected birds). This demonstrates that the cytokine storm which contributed to serious disease and mortality had been effectively controlled by the use of the mixture of flavonoids described herein. This data is consistent with a major reduction in IL-1 and IL-6 levels on administration of the nutritional supplement.

The reduction in TNFα levels by more than 90% is considered to indicate the desirability of use of the nutritional subject in patients infected by SARS-COV-2, including patients with diabetes, as unlike the use of dexamethasone, there does not appear to be concerns regarding glucose homeostasis. The nutritional supplement produced the reduction of TNFα when administered orally, which is an advantage when compared to other anti-TNF inhibitors such as antibodies, which require intravenous administration. Inhalation/insufflation to reach the lung is also contemplated.

Similarly, reduction of TGF-33 levels by over half is anticipated to be similarly beneficial in treatment of patients with hyperimmune reactions to SARS-COV-2. In addition, this reduction is anticipated to be beneficial at all stages of pneumonia by reduction of edema and fibrosis in the infected lung. Once again, oral administration of the nutritional supplement (or inhalation/insufflation to reach the lung) is more convenient than intravenous administration of biological agents.

This data also shows that, when treating a coronavirus infection, administering of the nutritional supplements herein from the early stages of SARS-COV-2 is effective in prevention or amelioration of hyperimmune conditions that can characterize serious late-stage disease.

### Example 6

### Tablet Formulation

An effervescent tablet formulation may be prepared on standard tabletting equipment using the following blended components:

Bioflavonoids 2 g of Reference Example 2

Tartaric acid 2 g

Sodium bicarbonate 2 g

(Stevia) 0.1 g

The sweetener (Stevia) was employed to render the solution obtained by dissolution of one or two tablets in water (200 ml) more palatable, as otherwise the bitter taste of the natural orange nutritional supplement is apparent. The sweetener employed is chosen to be naturally occurring so as to enhance the plant-derived nutritional supplement nature of the tablets, which can be used to provide solutions to be drunk to ameliorate the conditions referred to herein, such as those referred to in of Examples 1-5.

### Example 7

### Capsule Formation

A hard gelatine capsule dose was employed. Into this was placed 2 g of the mixture of Reference Example 2 to provide a unit dosage containing approximately 1 g of mixed bioflavonoids.

The capsule may be taken, for example, 1, 2, or 3 times a day to supplement the diet in order to treat conditions referred to herein, such as those referred to in Examples 1-4.

An analogous but smaller capsule form was prepared containing approximately 250-500 mg of bioflavonoids by hard filling capsules with 1 g or 500 mg of Reference Example 2 and hand closing the capsules. cl Example 8

### Prophylactic Use

A subject learned of possible infection with SARS-COV-2 via UK COVID Track and Trace. The subject employed the composition of Reference Example 4 by two administrations to each nostril from a multidose spray container. This was repeated 4 times a day for 5 days. The subject did not develop infection with SARS-COV-2.

1. A nutritional supplement for use to ameliorate the effect of a pneumonia in a human, which supplement comprises a mixture of bioflavonoids which contains at least 45% wt/wt (of the total bioflavonoids) of naringin and at least 15% wt/wt of neohesperidin, wherein said supplement is in a form suitable for oral administration or administration to the lung by insufflation or inhalation.

2. A nutritional supplement for use as claimed in claim 1, for the treatment of a coronavirus-caused pneumonia.

3. A nutritional supplement for use as claimed in claim 2, wherein the coronavirus is SARS-COV-2.

4. A nutritional supplement for use as claimed in any of claims 1-3, wherein the amelioration includes a reduction of an inflammatory condition caused by SARS-COV-2.

5. A nutritional supplement for use as claimed in any of claims 1-4, wherein the mixture of bioflavonoids comprises at least 70% wt/wt, for example, 80-90% wt/wt of naringin and neohesperidine.

6. A nutritional supplement for use as claimed in any of claims 1-5, wherein the mixture of bioflavonoids comprises bioflavonoids obtained from oranges, for example, the pith of bitter oranges, such as immature bitter oranges.

7. A nutritional supplement for use as claimed in any of claims 1-6 by administration by inhalation.

8. A nutritional supplement for use as claimed in any of claims 1-6 by administration orally.

9. A nutritional supplement for use as claimed in claim 8 or 9 for use in a human, where administration is in the form of a unit dose.

10. A nutritional supplement for use as claimed in claim 9, wherein dose is present in an insufflation device.

11. A nutritional supplement for use as claimed in claim 9, wherein the unit dose is in the form of a solid unit dose.

12. A nutritional supplement for use as claimed in any of claims 1-11 which further comprises a vitamin D, such as vitamin D3 or 25-hydroxyvitamin D3.

13. A nutritional supplement for use as claimed in any one of claims 1-12 which further comprises a cyclodextrin.

14. A nutritional supplement for use as claimed in any of claims 1-12, which further comprises a glycosaminoglycan.

15. A nutritional supplement for use as claimed in claim 13, wherein the glycosaminoglycan is hyaluronic acid.

16. A nutritional supplement for use to ameliorate a hyperimmune condition, which supplement comprises a mixture of bioflavonoids which contain at least 45% by weight (of the total bioflavonoids) of naringin and at least 15% wt/wt of neohesperidine.

17. A nutritional supplement for use as claimed in claim 16, wherein the hyperinflammatory condition occurs in the lung.

18. A nutritional supplement for use as claimed in claim 17, wherein the hyperinflammatory condition is as a result of a coronavirus infection.

19. A nutritional supplement for use as claimed in claim 18, for use in infection by SARS-COV-2.

20. A supplement for use as claimed in claim 16, wherein the hyperinflammatory condition occurs in a human suffering from sepsis or toxic shock syndrome.

21. A nutritional supplement for use as claimed in any of claims 15-19, wherein the supplement is as set forth in claim 1, 5 or 6.

22. A nutritional supplement adapted for administration by inhalation which comprises a mixture of bioflavonoids which contains at least 45% wt/wt (of total bioflavonoids) of naringin and at least 15% wt/wt of neohesperidine and an inhalable carrier therefor.

23. A device adapted to administer a composition to the lungs by inhalation which contains a composition as set forth in claim 22.

24. A nutritional supplement for use as claimed in any preceding claim, which further comprises biomass from oranges used as a source of the flavonoids.

25. A device for administering a mixture of flavonoids as set forth in any of claim 1, 5, 6, or 11-15, for use in treating SARS-COV-2 pneumonia in a human, which comprises a nebulizer containing said mixture of flavonoids, for example in aqueous solution.

26. An effervescent tablet or capsule which comprises a mixture of flavonoids as set forth in any of claim 1, 5, 6, or 11-15, and an efferent couple comprising a carbonate and/or bicarbonate and solid carboxylic acid.

27. An effervescent tablet as claimed in claim 26, wherein the carbonate or bicarbonate is a sodium oxide and/or potassium carbonate or bicarbonate.

28. An effervescent tablet as claimed in claim 26 or 27, wherein the carboxylic acid is citric acid, tartaric acid, malic acid and/or lactic acid.

29. An effervescent tablet as claimed in any of claims 26-28 for use in a human to ameliorate hypoxia in pneumonia.

30. An effervescent tablet as claimed in in claim 29, wherein the pneumonia results from a SARS-COV-2 infection.

31. A method of ameliorating a SARS-COV-2 pneumonia which comprises administration to the human in need thereof an effective amount of a nutritional supplement as set forth in any foregoing claim for prophylaxis of said infection, or for the treatment of said infection.

32. A method of ameliorating SARS-COV-2 pneumonia in a human which comprises administration by insufflation or inhalation to the lung of a nutritional supplement as set forth in any of claim 1, 5, 6 or 11-15.

33. A method as claimed in either of claim 31 or 32 for administration to a subject requiring administration of oxygen.

34. A method of ameliorating a cytokine storm caused by infection by a coronavirus such as SARS-COV-2, which comprises administering to the person in need thereof an effective amount of a nutritional supplement as set forth in any of claim 1, 5, 6 or 11-15.

35. A method as claimed in claim 34, wherein administration of the nutritional supplement is orally.

36. A method as claimed in claim 34, wherein administration of the nutritional supplement is to the lung, by inhalation or insufflation.

37. A method as claimed in any of claims 34-36, wherein the person suffering from SARS-COV-2 is hypoxic with SATS below 94.

38. A method as claimed in any of claims 34-36, wherein the patient has exhibited symptoms for at least 6 days.

39. A method of reducing the likelihood of a discharged patient requiring readmission to hospital, which comprises administering to the patient prior to and/or after discharge a nutritional supplement described in any foregoing claim.

40. A method as claimed in claim 39, wherein administration is oral and continues for at least 4 days post-discharge, for example at least 7, at least 10 or at least 21 days post-discharge.

41. A method for ameliorating adverse effects of SARS-COV-2 infection which persist after active viremia is brought under control, which comprises administration to the person infected by SARS-COV-2 a nutritional supplement as set forth in any foregoing claim, commencing at least 1, 2, 3 or 4 days after the first symptoms appear.

42. A nutritional supplement for use to treat or prevent SARS-COV-2 infection in the nasopharyngeal area of a human, which nutritional supplement contains at least 45% wt/wt (of the total bioflavonoids) of naringin and at least 13% wt/wt of neohesperdin, wherein said supplement is in a form suitable for intranasal administration.

43. A nutritional supplement for use as claimed in claim 42, wherein the mixture of bioflavonoids comprise at least 70% wt/wt, for example 80-90% wt/wt of naringin and neohesperidine.

44. A nutritional supplement for use as claimed in either of claim 42 or 43, wherein the mixture of bioflavonoids comprises bioflavonoids from oranges, for example the pith of bitter oranges, such as immature bitter oranges.

45. A nutritional supplement for use as claimed in any of claims 42-44, contained within a device which enables a spray of droplets to be introduced into the nasal cavity.

46. A nutritional supplement for use as claimed in claim 45, wherein the spray is composed of droplets sufficiently small to reach the anterior and posterior nasal cavities.

47. A nutritional supplement for use as claimed in any of claims 42-46, which is an aqueous solution.

48. A nutritional supplement for use as claimed in any of claims 42-47, which further comprises at least one polysaccharide which assists in immobilizing or inhibiting SARS-COV-2.

49. A nutritional supplement for use as claimed in claim 48, wherein the polysaccharide is a glycosaminoglycan.

50. A nutritional supplement for use as claimed in claim 49, wherein the glycosaminoglycan is

51. A nutritional supplement for use as claimed in claim 48, wherein the polysaccharide is a cyclodextrin, such as betacyclodextrin or hydroxypropyl cyclodextrin.

52. A nutritional supplement for use as claimed in claim 48, wherein the 52. polysaccharide is a sulfated polysaccharide.

53. A nutritional supplement for use as claimed in claim 52, wherein the sulfated polysaccharide is a carrageenan, preferably iota carrageenan, or is a sulfated glycosaminoglycan, such as chitosan.

54. A nutritional supplement for use as claimed in any of claims 42-53 which further comprises glycerol.

55. A nutritional supplement for use as claimed in any of claims 42-54 which further comprises a buffer.

56. A nutritional supplement for use as claimed in any of claims 42-55 which has a pH of 3-8, for example 3.5-7.

57. A nutritional supplement for use as claimed in any of claims 42-56, wherein the pH is 3-4.5, for example 3.5.

58. A nutritional supplement for use as claimed in any of claims 42-57, wherein the pH is 6.5-8, for example 7-7.5.

59. A nutritional supplement for use as claimed in any of claims 42-58 which comprises one or more carboxylic acids, such as citric acid, tartaric acid or lactic acid.

60. A nutritional supplement for use as claimed in any of claims 42-59 which further comprises xylitol, for example 3-30% wt/wt, such as 5-10% wt/wt.

61. A nutritional supplement for use as claimed in any of claims 42-60 which further comprises a nitrite, such as sodium nitrite and potassium nitrite.

62. A nutritional supplement for use as claimed in any of claims 42-61 to reduce likelihood of an infected person transmitting SARS-COV-2.

63. A nutritional supplement as claimed in any of claims 42-61 for use in reducing the chance of an uninfected person catching SARS-COV-2.

64. A nutritional supplement for use as claimed in any of claims 42-61 for administration to a person having a nasopharyngeal infection with SARS-COV-2 to reduce transmission of the virus to the lower respiratory tract.

65. A nutritional supplement for use as claimed in any of claims 42-61 to aid in the restoration of a lost sense of smell, or to reduce the likelihood of developing a reduced sense of smell as a result of SARS-COV-2 infection.

66. A nutritional supplement for use as claimed in any of claims 42-65 which comprises a 0.25% wt/wt to 7.5% wt/wt, for example 0.5% wt/wt to 5% wt/wt, such as 1% wt/wt to 2.5% wt/wt, of the mixture of bioflavonoids.

67. A nutritional supplement for use as claimed in any of claims 42-66 which is isotonic.

68. A nutritional supplement as set forth in any foregoing claim for use in the amelioration of edema and/or fibrosis of the lung caused by SARS-COV-2 infection.

69. A nutritional supplement for use as claimed in claim 68 for oral administration.

70. A method of ameliorating edema and/or fibrosis in a subject suffering from SARS-COV-2 infection which comprises the administration of an effective amount of a nutritional supplement as set forth in any foregoing claim to a subject in need thereof.

71. A method according to claim 70, wherein administration is oral.

72. A nutritional supplement for use or a method as claimed in any previous claim, wherein the recipient of the flavonoids is over 50 years old, over 60 years old, over 70 years old or over 80 years old.

73. A nutritional supplement for use or a method as claimed in any previous claim, wherein the recipient of the flavonoids has a chronic heart condition, a chronic liver condition, a chronic kidney condition, diabetes, or a BMI of over 30, over 35 or over 40.

74. A nutritional supplement for use to a method as claimed in any previous claim, wherein the recipient of the flavonoids has type II diabetes.

75. A nutritional supplement for use as claimed in claim 1, for the treatment of a bacterial pneumonia.

76. A nutritional supplement for use as claimed in claim 75 wherein the bacteria is Streptococcus pneumoniae.
